(19) United States
(12) Patent Application Publication
(10) Pub. No.: US 2026/0256702 A1
(43) Pub. Date: Sep. 3, 2026

(54) SUSTAINED ADJUNCT THERAPY TO IMPROVE CHEMOTHERAPY EFFICACY IN GLIOBLASTOMA IN A CEREBROVASCULA TUMOR-ON-A-CHIP MODEL

(71) Applicant: Ohio State Innovation Foundation, Columbus, OH (US)

(72) Inventors: Aleksander Skardal, Columbus, OH (US); Katelyn Reilly, Columbus, OH (US); Rengasayee Veeraraghavan, Columbus, OH (US)

(21) Appl. No.: 18/873,010

(22) PCT Filed: Jun. 9, 2023
(86) PCT No.: PCT/US2023/068162

§ 371 Date: Dec. 9, 2024

Related U.S. Application Data
(60) Provisional application No. 63/366,199, filed on Jun. 10, 2022.

(51) Int. Cl. A61K 9/50; A61K 31/495; A61K 38/08; A61P 35/00

(52) U.S. Cl. CPC A61K 9/5073; A61K 9/5089; A61K 31/495; A61K 38/08; A61P 35/00

(57) ABSTRACT

A cerebrovascular unit tumor (CVU-T)-on-a-chip model that models both the blood brain barrier (BBB) and tumor is described. The model provided a physiologically relevant GBM tumor model including a corresponding microenvironment complete with a BBB component that is crucial in dictating drug bioavailability to the tumor. Additionally a novel biodegradable microcapsule for sustained local delivery of a therapeutic such as a connexin hemichannel inhibitor is provided. The therapeutic sensitizes malignant GBM populations to systemic TMZ chemotherapy.

### CROSS-REFERENCE TO RELATED APPLICATIONS

This application claims the benefit of U.S. Provisional Application No. 63/366,199, filed Jun. 10, 2023, which application is incorporated herein by reference in its entirety.

### GOVERNMENT FUNDING

The present invention was made with government support under Grant No. R21CA229027 awarded by the National Institutes of Health. The US government has certain rights in this invention.

### FIELD

This invention relates to an implantable microcapsule for delivery of a therapeutic unable to cross the blood brain barrier and a model for testing glioblastoma therapeutic treatment.

### BACKGROUND

Glioblastoma (GBM), the most common primary brain tumor, has dismally low long-term survival. Despite aggressive treatment, involving surgical resection followed by chemo/radiotherapy, recurrence is nearly universal and the 5-year overall survival rate remains at only 5% with most tumors recurring within a year. Tumor recurrence in GBM patients is universal despite aggressive surgery and chemo/radiotherapy. Half a century of research has only increased average life expectancy by <3 months with just four new treatments gaining FDA approval. New therapies are urgently needed.

In GBM, critical challenges derive from the limited range of drugs that can permeate the blood brain barrier (BBB; such as temozolomide [TMZ]), and the heterogeneous and dynamic nature of GBM tumors. GBM is comprised of distinct subpopulations that evolve, both under native conditions and in response to therapies, thereby confounding treatment. Importantly, prevalence of a glioma stem cell (GSC) subpopulation correlates with poor prognosis due to their ability to evade chemotherapy. GSCs, as well as some additional GBM subpopulations, may do so by drug efflux machinery—using connexin43 (Cx43) hemichannels to pump TMZ out of the cell. Cx43 hemichannel inhibitors, such as αCT1 and αCT11, can sensitize GSCs to TMZ in vitro, but they cannot cross the BBB, necessitating local delivery at the tumor site sustained over extended periods (months). Previous nanoparticle-based approaches have been limited to 2-3 weeks.

### SUMMARY

A bioengineered three-dimensional glioblastoma model for testing glioblastoma therapeutics is described. The model includes a blood brain barrier; a neural compartment, a brain parenchyma; and a tumor organoid, thus permitting both implants and crossing of the blood brain barrier to be evaluated. Use of the model then permits for evaluation of methods such treating glioblastoma in a subject. In this case, coincident with surgical glioblastoma removal from the subject, the method includes a step of providing a biodegradable microcapsule device loaded with a connexin hemichannel inhibitor together with treatment of a therapeutic that crosses the blood brain barrier. In some aspects, the invention includes a biodegradable microcapsule device and methods of making the microcapsule.

Other methods, features and/or advantages is, or will become, apparent upon examination of the following figures and detailed description. It is intended that all such additional methods, features, and advantages be included within this description and be protected by the accompanying claims.

### BRIEF DESCRIPTION OF THE DRAWINGS

FIG. 1: Schematic describing the biological structure of the CVU-T, systemic TMZ delivery, and microcapsule-based αCT11 delivery; and the mechanism of αCT11-based Cx43 hemichannel inhibition of temozolamide(TMZ) efflux.

FIG. 2A-2C: ECM composition modulates BBB transport. 2A) Our ECM hydrogel, comprised of HA, gelatin (or collagen), and PEG-based crosslinkers (to modulate elastic modulus), are supplemented with proteins or derivative peptides, such as lab-modified FN and LMN. Addition of FN/LMN results in 2B) robust EC layers expressing tight junction biomarkers (ZO-1), and C) control over molecular weight-based mass transport selectivity. Only with both FN and LMN present, can the BBB completely prevent 3-5 kDa MW FITC-dextran passage.

FIG. 3A-3C: Drug screening in PTOs from a responsive patient. 3A-3C) PTO response to TMZ changes after cells have been cultured in 2D on tissue culture plastic. 3A) Freshly made organoids (no passaging—P0). 3B) PTOs made after 1 passage (P1) and 3C) 2 passages (P2). PTOs gain resistance to TMZ over time in 2D. *p<0.5 vs. control.

FIG. 4: HA-based ECM hydrogels preserve genomic profiles of GBM biospecimen-derived cells. RNAseq cluster analysis of originating tumor cells (Tu), organoids (Org), temozolomide-treated organoids (Org_T), and cells maintained in 2D tissue culture (Pla), from 6 human patients (A, C, E, F, G, and H). Tu, Org, and Org_T cluster together by patient, while Pla cultures cluster together, regardless of patient. Org/Org_T culture preserved the initial majority GBM subtype, while Pla promotes the mesenchymal subtype.

FIG. 5A-5D: Microcapsules for sustained release. 5A) SEM images of sample hollow capsule (scale bar 100 μm), and nanopores in the outer layer of capsules produced with 5, 7.5, and 10% salt concentrations (scale bar 5 μm). 5B-5C) In vitro release of BSA (5B) and anti-VEGF (5C) from capsules over 9-12 months, with extended release from bilayered capsules and capsules with smaller pores. Lower MW BSA was released more rapidly than anti-VEGF from capsules with the same design and porosity (same line colors). 5D) 5 ng VEGF was used to induce angiogenesis in HUVECs, as seen by green calcein AM stain in the control. There was no significant difference in tubule size between cells treated with anti-VEGF (bevacizumab) released after 9 months (far right) and fresh anti-VEGF control (middle), indicating preservation of bioactivity.

FIG. 6A-6C: αCT11 sensitizes TMZ-resistant organoids and can be controllably delivered. Addition of αCT11 to 6A) U87 MG GBM organoids makes little impact, while 6B) it sensitizes more resistant A172 GBM organoids to TMZ. 6C) αCT11 release rate from microcapsule embedded in hydrogel is significantly decreased compared to therapeutic directly loaded into hydrogel for at least 3 weeks (n=4). Statistics: *p<0.05 compared to control; #p<0.05; † p<0.01 between groups at each timepoint.

FIG. 7A-7D: CVU device design. 7A) The CVU uses 3 sets of corresponding media reservoirs to drive fluid flow. The device seen from above (scale bar—1 cm), and 7B) a cross section showing the tubular BBB organization within the ECM hydrogel and cells populating the 3D environment. Cell density is higher than depicted. 7C) DLP biofabrication is used to build multiple lumen-containing, high resolution CVU structures in parallel. The laser projector controls the patterning of crosslinking which changes as the build platform travels up out of the bioink volume. (i) Inset: A DLP bioprinted CVU structure. 7D) Flow of cells through the central lumen (L) within the parenchymal walls.

FIG. 8A-8B: Representative 3D sDCI images. 8A) Intercalated disk (sites of cell-cell contact) from a mouse heart labeled for gap junction (connexins 40, 43), mechanical junction (N-cadherin) and ion channel (NaV1.5) proteins. 8B) Accumulation of active (βadp1) but not inactive peptide drug (βadp1-scr) at intercalated disks from peptide-treated mouse hearts. βadp1 is preferentially enriched near Cx43, where its target NaVβ1 is localized.

FIG. 9A-9C: 9A) τSTED images of murine intercalated disks show Na V1.5 sodium channels closely associating with Cx43 gap junctions. 9B) OBS3D analysis provides a quantitative picture of molecular organization in the form of bivariate histograms of sodium channel cluster mass vs. distance from Cx43 clusters (gap junctions). 9C) Enrichment ratios quantify the relative density of NaV1.5 channels near (<100 nm from cluster edges) Cx43 gap junctions [n=3 hearts/group, 5 images/heart; *p<0.05 vs. control, Wilcoxon's test].

FIG. 10A-10E: Representative STORM single molecule localization images showing NaV1.5 sodium channels and Cx43 gap junctions in murine hearts treated with 10A) vehicle (control) or 10B) VEGF (100 pg/ml, 60 minutes). STORM-RLA provides 10C, 10D) bivariate plots of NaV1.5 cluster density vs. distance from Cx43 clusters as well as 10E) simple indices such as the % of NaV1.5 molecules near Cx43 (<100 nm from cluster edge).

FIG. 11A-11E: Tunable capsules are prepared as shown. 11A) The chitosan inner layer and PCL outer layer are collected as nanofibers on a small rod by electrospinning. 11B) The electrospun material is sintered in a vacuum oven to remove macroscale pores. 11C) Rod removal results in a hollow cylinder. 11D) Salt leaching leaves nanoporous structure in the outer PCL layer. 11E) Therapeutic is loaded into the center of the hollow cylinder and ends are sealed, resulting in a sealed microcapsule

FIG. 12A-12F: αCT1, not αCT11, enhances TMZ efficacy in BT169 GSC organoids and heterogeneous GBM PTOs. 12A) In BT169 GSC cells, αCT11 fails to sensitize to TMZ, while 12B) αCT1 does. 12C) a non-GSC GBM cell line, such as A172, does not benefit from αCT1. 12D) LIVE/DEAD staining supporting BT169 treatment with αCT1/TMZ in 12B). Green—Calcein AM-stained viable cells; Red—ethidium homodimer-1-stained dead cell nuclei. 12E-12F) Testing of αCT1/TMZ combinatorial treatment in heterogeneous patient-derived GBM organoids indicates that αCT1 enhances TMZ efficacy. Statistical significance: *p<0.05; **p<0.01.

### DETAILED DESCRIPTION

We provide a novel biodegradable microcapsule for sustained local delivery of a connexin hemichannel inhibitor, such as αCT11 or alternatively αCT1 or other Cx43 inhibitors, to sensitize malignant GBM populations to systemic TMZ chemotherapy. Microcapsules containing a connexin hemichannel inhibitor, such as αCT11 are implanted on the brain parenchymal side of the CVU-T, mimicking standard-of-care placement of Gliadel wafers immediately following surgery. TMZ and other relevant compounds are administered systemically, as in the clinic, and assessed for BBB transport and tumor killing efficacy.

The novel methods are validated using a cerebrovascular unit tumor (CVU-T)-on-a-chip model that models both the BBB and tumor. The CVU-T advances current BBB models, which are largely planar, static, or both, to a tubular platform comprised of digital light processing (DLP) bioprinted brain ECM-mimicking hydrogel containing a variety of neural cell types. GBM tumor organoids, derived from glioma patient tumor biospecimens are embedded within the brain parenchyma of a CVU-T device, thus providing a physiologically relevant GBM tumor model including a corresponding microenvironment complete with a BBB component that is crucial in dictating drug bioavailability to the tumor. To bolster these efforts, in addition to a portfolio of chemical and biological assays, super-resolution microscopy-based technologies enable 3D mapping of individual cell types within CVU-Ts and the localization of 1) key protein targets (e.g. Cx43) relative to molecular landmarks and 2) fluorescently tagged drug compounds to directly validate drug delivery to the molecular target.

In one aspect, a bioengineered three-dimensional glioblastoma model for testing glioblastoma therapeutics is provided. The model including a blood brain barrier; a neural compartment, a brain parenchyma; and a tumor organoid. In some aspects, the tumor organoid is patient derived. In some aspects, the tumor organoid includes at least one of the following: astrocytes, pericytes, microglia, oligodendrocytes, neurons, and glioblastoma cells. In some aspects the tumor organoid includes all of the following: astrocytes, pericytes, microglia, oligodendrocytes, neurons, and glioblastoma cells.

In some aspects, the invention includes a method for treating glioblastoma in a subject. In the method, coincident with surgical glioblastoma removal from the subject, the method includes a step of implanting in a tumoral area a biodegradable microcapsule device loaded with a connexin hemichannel inhibitor. The microcapsule providing sustained release of a connexin hemichannel inhibitor to the tumoral area for a period of time that is at least two months. The method further including post-surgical treatment with a therapeutic that crosses the blood brain barrier. In some aspects the therapeutic is TMZ. In some aspects, the microcapsule continues release of the connexin hemichannel inhibitor continuously for six months. In some aspects, the connexin hemichannel inhibitor is αCT11.

In some aspects, the invention includes a cylindrical shaped biodegradable microcapsule device. The device including: a core comprising a connexin hemichannel inhibitor; an inner layer of chitosan nanofibers surrounding the core; an outer layer of PCL nanofibers surrounding and adhered to the inner chitosan layer; nanopores throughout the microcapsule; and where each end of the microcapsule is. In some aspects, the microcapsule includes in the core the connexin hemichannel inhibitor αCT11. In some other aspects, the microcapsule includes in the core the connexin hemichannel inhibitor αCT1. In some aspects, the microcapsule is characterized by more than 100 nanopores, each nanopore between about 10 and about 1000 nm in diameter, the microcapsule being approximately 5 to 20 mm in length with a diameter of about 0.5-2 mm.

In some aspects, the invention includes methods of making a microcapsule. The method may include steps of: providing a rod; making an inner layer by collecting chitosan nanofibers on the rod by electrospinning; making an outer layer by collected PCL nanofibers on the chitosan inner layer by electrospinning; sintering the electrospun materials to remove macroscale pores; removing the rod creasing a hollow cylinder; salt leaching the hollow cylinder to create a nonporous structure; sealing a first end of the hollow cylinder; inserting a therapeutic into the hollow cylinder; sealing the second end, thereby enclosing the therapeutic within the cylinder and forming a microcapsule.

### Definitions

As used herein and in the appended claims, the singular forms “a”, “and”, and “the” include plural referents unless the context clearly dictates otherwise. Thus, for example, reference to “a sample” also includes a plurality of such samples and reference to “the splicing regulator protein” includes reference to one or more protein molecules, and so forth.

As used herein, the term “about” refers to +/−1% deviation from the basic value.

As used herein, the term “tumor” refers to any neoplastic growth, proliferation or cell mass whether benign or malignant (cancerous), whether a primary site lesion or metastases.

As used herein “therapeutically effective amount” refers to an amount of a composition that relieves (to some extent, as judged by a skilled medical practitioner) one or more symptoms of the disease or condition in a mammal. Additionally, by “therapeutically effective amount” of a composition is meant an amount that returns to normal, either partially or completely, physiological or biochemical parameters associated with or causative of a disease or condition. A clinician skilled in the art can determine the therapeutically effective amount of a composition in order to treat or prevent a particular disease condition, or disorder when it is administered, such as intravenously, subcutaneously, intraperitoneally, orally, or through inhalation. The precise amount of the composition required to be therapeutically effective will depend upon numerous factors, e.g., such as the specific activity of the active agent, the delivery device employed, physical characteristics of the agent, purpose for the administration, in addition to many patient specific considerations. But a determination of a therapeutically effective amount is within the skill of an ordinarily skilled clinician upon the appreciation of the disclosure set forth herein.

Treat”, “treating”, and “treatment”, etc., as used herein, refer to any action providing a benefit to a patient at risk for or afflicted with a disease, including improvement in the condition through lessening or suppression of at least one symptom, delay in progression of the disease, prevention or delay in the onset of the disease, etc. Treatment also includes partial or total destruction of the undesirable proliferating cells with minimal destructive effects on normal cells. A subject at risk is a subject who has been determined to have an above-average risk that a subject will develop cancer, which can be determined, for example, through family history or the detection of genes causing a predisposition to developing cancer.

The term “subject,” as used herein, refers to a species of mammal, including, but not limited to, primates, including simians and humans, equines (e.g., horses), canines (e.g., dogs), felines, various domesticated livestock (e.g., ungulates, such as swine, pigs, goats, sheep, and the like), as well as domesticated pets and animals maintained in zoos.

### Microcapsule

The microcapsule described herein may be of any size or shape. Pore size as well as the total number of pores may be varied as needed to deliver successfully uniformly the therapeutic enclosed within the microcapsule. Likewise, the microcapsule may contain more than one therapeutic.

The microcapsules described herein are advantageous in that they may be modified to fine tune medication or adjuvant delivery to a site. This fine tuning may be adjustable depending upon the subject, the type and location of a site to be treated, such a tumor location, or other conditions.

Thus in one aspect, a microcapsule having a tubular shape with two ends that are closed, wherein each of the one or more capsules independently comprises a multi-layered wall and at least one luminal compartment; and one or more therapeutic agents each initially present within one or more of the at least one luminal compartments. Each multi-layered wall independently comprises at least an inner-layer and an outer layer and each inner layer comprises a first polymer having a net positive charge under physiological conditions and each out layer independently comprises a second polymer that differs from the first polymer.

In some aspects, the drug delivery composition may comprise two or more capsules. In some embodiments, a different therapeutic agent is initially present within each of the two or more capsules. In other embodiments, the same therapeutic agent is initially present within each of the two or more capsules.

In some aspects, at least one or the one or more capsules comprises two or more luminal compartments. In some embodiments, a different therapeutic agent is initially present within each of the two or more luminal compartments.

In other aspects, the same therapeutic agent is initially present within each of the two or more luminal compartments.

In some aspects, a first polymer, or the polymer layer forming an inner layer, or a layer enclosing the luminal core compartment may include a chitosan nanopolymer, chitosan, a polyethyleneimine, a protamine, a polypropylimine, a poly-L-lysine, a poly-L-arginine, a poly-D-lysine, a poly-D-arginine, a cellulose, a dextran, a poly(amidoamine), poly(2-(dimethylamino)ethyl methacrylate), derivatives thereof, or combinations thereof. In some embodiments, the first polymer may comprise a chitosan or derivatives thereof.

In some aspects, a second polymer, or the outer layer, or a layer adhered to the inner layer may be a biodegradable polymer. In some embodiments, this second polymer layer may be PCL nanofibers, a poly(e-caprolactone) (PCL), a poly-lactic acid (PLA), a poly-glycolic acid (PGA), a poly-lactide-co-glycolide (PLGA), a polyester, a poly(ortho ester), a poly(phosphazine), a poly(phosphate ester), a gelatin, a collagen, a polyethylene glycol (PEG), derivatives thereof, or combinations thereof. In some aspects, the second polymer comprises PCL. In other aspects, the second polymer comprises PLA.

In some aspects, all edges of the microcapsule are necessarily sealed or enclosed so as to prevent leakage of the therapeutic enclosed in the luminal core compartment from exiting the microcapsules by any means other than the pores. Thus “edges” refers to any surface where the luminal compartment meets the polymer layers of the device or the multiple polymer layers meet each other that are necessary to seal off or enclose so that the final microcapsule product contains no means of the enclosed therapeutic to exit the microcapsule other than the pores.

In some aspects, the microcapsules described herein are used for any use, which may or may not exclude ocular administration of a therapeutic.

### Applications of the Microcapsule

The microcapsules described herein have wide applicability to delivery of medication or adjuvant to a surrounding tissue environment in numerous applications.

The microcapsules described herein may be implanted in or proximal or adjacent to any tissue that is to be treated. The microcapsules may be administered coincident with surgery, or implanted subcutaneously, have wide applicability to delivery of medication or adjuvant to a surrounding tissue environment in numerous applications.

### Models

In some aspects a 3-D CVU-T model may be used for determination of the effectiveness of combinations of therapeutics and microcapsules containing a medication or adjuvant. It is appreciated however, that the aspects of the invention for evaluating the effectiveness of this combinatorial therapy may be applied to any 2-D, in vitro or in vivo model.

### Therapeutics, Medications and Adjuvants

In some aspects the invention includes deliver of a medication or adjuvant through extended release from an implanted microcapsule together with additional administration of a therapeutic by another means. It is appreciated that any therapeutic, medication or adjuvant may be applied in any combinations. That is, in some instances a chemotherapeutic agent and an adjuvant are both presented to an area to be treated in the form of a microcapsule. In some instances a microcapsule that delivers a medication or adjuvant is combination with other treatments such as immunotherapy, cellular therapy, spherical nucleic acids, oscillating magnetic field, sonication or a combination of therapies.

In some aspects of the invention, the therapeutic temozolamide is combined with an adjuvant that is a connexin hemichannel inhibitor for treatment of a glioblastoma. It is appreciated that the methods according to many aspects of the invention may be practiced with any glioblastoma therapeutic such as MGMT inhibitors, HDAX and FAO inhibitors, BET inhibitors, P13K/AKT/mTOR pathway inhibitors, interferon inhibitors, bevacizuman, paxalisib, O₆BG, or any combination of therapeutics. Likewise if the methods are directed to treatment of a cancer or condition other than glioblastoma, any appropriate therapeutic may be applied. The therapeutic may be applied in combination with a microcapsule loaded with the appropriate medication or adjuvant or in therapeutic may be present in the microcapsule.

According to aspects of the invention, the connexin hemichannel inhibitor may serve as a therapeutic, which is a treatment, therapy or drug, or adjuvant, which is a substance for enhancing an immune response or enhancing the effect of a therapeutic, for any disease, disorder or condition.

### Connexin Hemichannel Inhibitors

In some aspects of the invention a connexin hemichannel inhibitor as an adjuvant is described. The connexin hemichannel inhibitor may be a peptide mimetic or any other appropriate connexin hemichannel inhibitor. While some aspects of the invention describe the use of αCT11 or αCT1, it is appreciated that any connexin hemichannel inhibitor may be loaded into the microcapsules according to the methods of the invention.

In some aspects of the invention a connexin hemichannel can be a connexin Cx43, Cx32, Cx46, Cx37, Cx40, Cx50, Cx59, Cx62, Cx26, Cx31, Cx30.3, Cx31.1, Cx30, Cx25, Cx45, Cx47, Cx30.2, Cx36, Cx31.9, Cx39, Cx40.1, Cx23, or Cx29 hemichannel. In certain aspect the hemichannel is a Cx43. In some aspects, the compositions and methods of the invention are applicable to pannexins. Further any pharmacological tools can be used to block hemichannels is applicable to the methods and composition described herein. In some aspects, an inhibitor may be a αCT11, αCT1, amitriptyline, or any connexin-blocking agents.

Unless defined otherwise, all technical and scientific terms used herein have the same meaning as commonly understood by one of ordinary skill in the art to which this invention belongs.

### EXAMPLES

### Example 1—Development of a Cerebrovascular Unit Tumor (CVU-T)-on-a-Chip Model

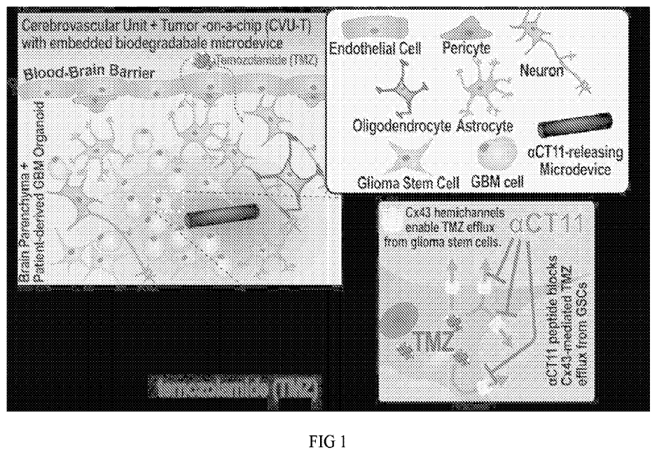

Referring to FIG. 1, a schematic describing the biological structure of the CVU-T, systemic TMZ delivery, and microcapsule-based αCT11 delivery is shown; and the mechanism of αCT11-based Cx43 hemichannel inhibition of TMZ efflux.

A 3D cellularized system models are still relatively rare. When combined with microfluidic device housings as described herein, these models address the shortcomings of previous planar and spheroid BBB models. We have advanced the complexity of the model system from simple spheroids to ECM hydrogel-supported tissue and tumor organoids—including patient-derived organoids that have undergone a variety of drug screening studies—and more complex “body-on-a-chip” platforms consisting of up to six organs/tissues on a chip. Unlike most other BBB platforms, our CVU enables a platform with multiple sampling points, compatibility with onboard sensing, while also using a 3D architecture of the CVU to mimic the blood-brain interface (FIG. 1).

We deploy a CVU-Tumor (CVU-T) tissue chip to test a combinatorial TMZ-αCT11 therapy, utilizing a bioengineered microcapsule-based drug delivery system to be implanted in the brain similar to standard-of-care placement of Gliadel wafers following surgical debulking of the tumor. The αCT11-loaded microcapsules are implanted on the brain parenchymal side of the CVU-T, while systemically administering TMZ, and use super-resolution microscopy to validate payload delivery to mechanistic targets (FIG. 1).

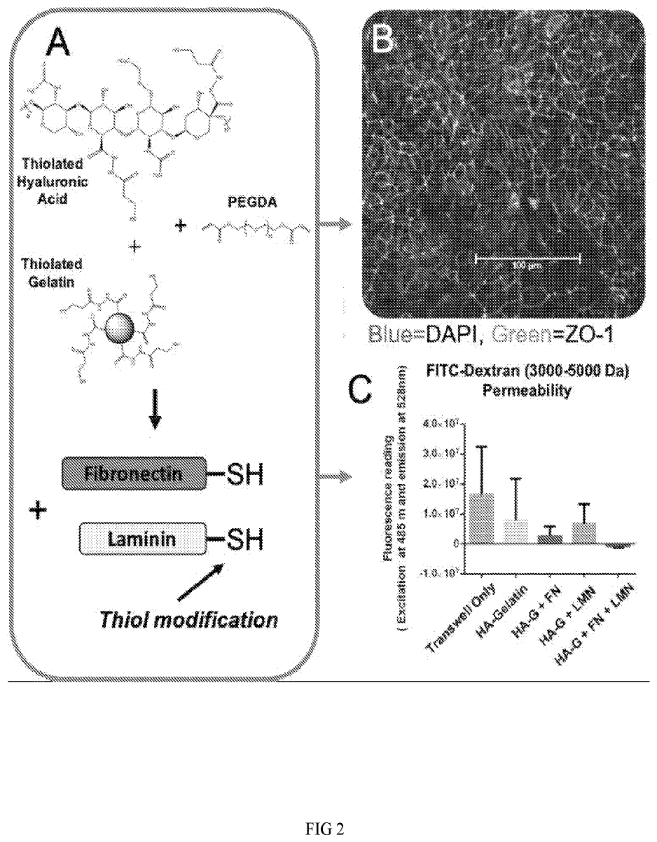

Referring to FIG. 2, a hydrogel formulation approach in which specific synthetically modified adhesion proteins, such as FN and LMN, can be covalently tethered to HA and collagen or gelatin base components (FIG. 2A). By modulating the ratios of the adhesion proteins, we could drive a tight brain microvascular endothelium monolayer, as visualized by zonula occludens-1 (ZO-1) immunofluorescence (FIG. 2B), and increased barrier function of this simplified BBB system. Barrier function is shown via trans-endothelial electrical resistance (TEER) sensing (FIG. 2C).

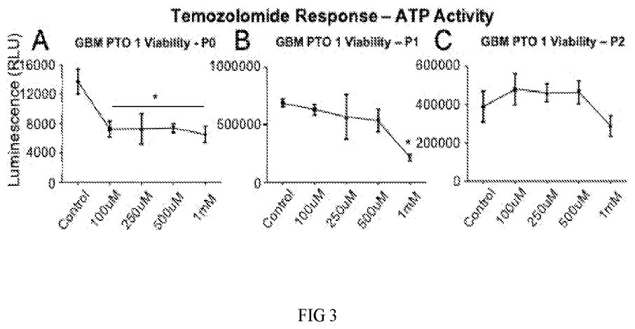

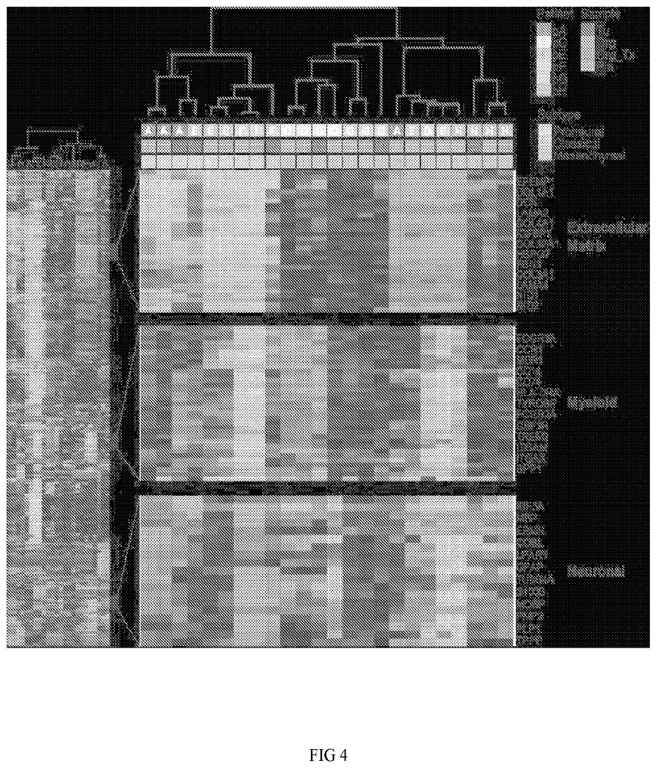

In parallel, we have established patient tumor organoids (PTOs) from a range of tumor types. These PTOs have been deployed in chemotherapy and immunotherapy studies. For example, FIG. 3 shows glioma PTO response to TMZ. Importantly, PTOs made directly from fresh tumor biospecimens showed TMZ sensitivity and tumor cell death. However, if the cells underwent just one or two passages in 2D culture, even if returned to 3D PTO form, they gained TMZ resistance. This suggests that the artificial nature of 2D culture drives a shift in phenotype and/or genotype. To further understand this shift, we analyzed RNA sequencing (RNAseq) data from biospecimens, 3D PTOs, TMZ-treated PTOs, and 2D cultured cells from 6 glioma patients. Cluster analysis of the 1000 most variably expressed genes resulted in clusters in which the original biospecimens, 3D PTOs, and TMZ-treated PTOs clustered together by patient, while in general the 2D cultured cell populations clustered together, while shifting to the mesenchymal subtype (FIG. 4). This RNAseq data strongly indicates that our 3D ECM hydrogel organoid system maintains the genetic profile of the originating tumor, and more accurately represent the diseases of individual patients.

### Example 2: Microcapsule Technology

We have developed a novel process that combines electrospinning, sintering, and salt leaching to create tunable capsules for long-term controlled release of a compound. The capsules enable high levels of therapeutic loading with preserved bioactivity due to the hollow core that is loaded post-processing without the typical loss seen during encapsulation procedures. The polymers in the device are biocompatible, biodegradable, and used for applications in drug delivery. Chitosan, a positively charged biopolymer was selected for the inner layer of the capsule to electrostatically interact with negatively charged proteins, peptides, and antibodies (e.g. anti-VEGF, αCT11) to slow release. Poly(caprolactone) (PCL) was selected for the outer layer of the device due to its hydrophobicity that slows hydrolytic degradation. Capsule inner diameters ranged from 0.26-1.7 mm, with average wall thickness of 90 μm. Scanning electron microscopy (SEM) was used to measure capsule dimensions and pore size (FIG. 5A). No pores were present after sintering when prepared without salts. Pore size increased from 237±97 nm to 609±274 nm for 1% to 10% salt concentrations, and elution was increased at >5% salt concentration due to pore interconnectivity. In preliminary studies, in vitro release of model protein BSA and anti-VEGF (bevacizumab) were evaluated. Capsules sustained release of BSA (molecular weight [MW] 66.5 kDa) at least 12 months and anti-VEGF (149 kDa) at least 9 months, as determined by BCA and enzyme-linked immunosorbent assays (ELISA). Therapeutic was not completely released from some capsules at the end of the study (FIG. 5B-5C), demonstrating potential for release beyond 12 months. In vitro bioactivity of bevacizumab eluted from capsules was validated by capillary tubule formation assay using human umbilical vein endothelial cells (HUVECs) (FIG. 5d). HUVECs were exposed to VEGF at 5 ng/ml mixed with 10 μg/ml native bevacizumab control and 10 μg/ml bevacizumab released from capsules at time points out to 9 months. As shown by calcein AM staining, tubule formation was inhibited, demonstrating therapeutic bioactivity after long-term release.

### Example 3: TMZ-αCT11 Combinatorial Treatment in GBM Cell Line Organoids and αCT11 Microcapsule Release

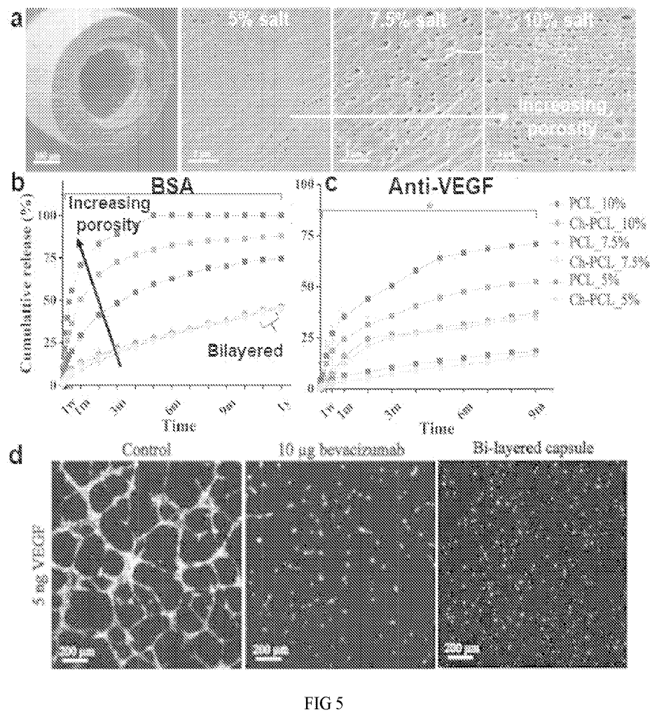

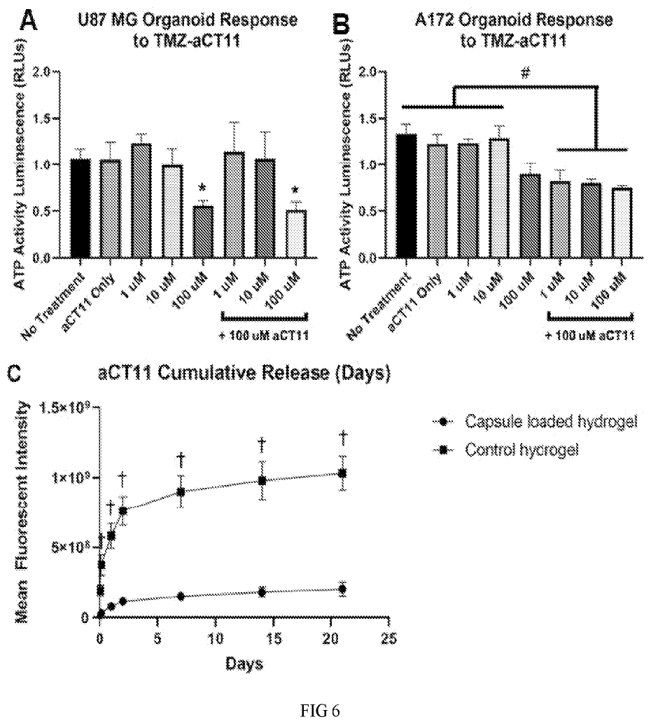

TMZ and αCT11 have been tested together against GBM cells in simple 2D cell cultures with a single GBM cell line, demonstrating improved TMZ efficacy. To build on these 2D studies we created GBM organoids from the cell lines U87 MG and A172, and assessed whether or not αCT11 improved TMZ efficacy in 3D. FIG. 6 shows organoid responses via quantitative ATP activity following 8 days of treatment. In U87 MG organoids, which responded readily to TMZ, addition of αCT11 made little difference (FIG. 6A). However, A172 organoids were largely resistant to TMZ, except at the highest dose, while αCT11 increased TMZ efficacy even at low doses (FIG. 6B), indicating that αCT11 improves TMZ efficacy in TMZ-resistant cells in 3D. Lastly, we undertook release studies for preliminary evaluation of our microcapsule technology to sustain long-term release of αCT11, which has a lower MW relative to previously evaluated therapeutics. Over 21 days, αCT11-loaded microcapsules, embedded in our HA ECM hydrogel, minimized the initial burst release and supported a measurable, but significantly reduced sustained release of αCT11 compared to αCT11 loaded directly into the hydrogel without the microcapsule (FIG. 6C).

### Example 4: Optimization and Characterization of a Locally Defined DLP Bioprinted CVU-Tumor Device

The CVU platform through modulation of ECM components and incorporation of primary and iPSC-derived neural cells and integrated GBM PTOs will fully realize the CVU-T system. Super-resolution microscopy techniques validate cell and tissue architecture and spatial resolution of molecular targets for drug studies in subsequent examples.

### Biofabrication of CVU-T

Neural cell cultures. Cells will be generated for each lineage using iPSCs (WiCell) ECs: iPSCs will be seeded and differentiated for 4 days in defined E6 medium. Impure immature cells are treated for 2 days with EC medium supplemented with bFGF and retinoic acid (RA). Mature brain microvessel endothelial cells (BMECs) are purified for 24 h in EC medium supplemented with bFGF and RA. 24 h after purification, barrier phenotype will be induced by treating cells with EC medium lacking bFGF and RA. Neural progenitor cells (NPC): This protocol builds on prior protocols. iPSC cultures are grown to 70% confluence then mTeSR1 is replaced with neural induction medium (Advanced DMEM: F-12 with Glutamax and 2% Neurobrew21) with 1 μM IWR1 until day 5 and dual SMAD inhibition until day 10. On day 10, confluent cultures are treated with 10 μM Y-27632 for 1 hour before dissociation with Accutase. Cells are plated onto fresh laminin-coated plates, with a split ratio of 1:2 in Advanced DMEM: F-12 (with Glutamax). NPCs at this stage are strongly positive for PAX6, SOX2, FOXG1. Astrocyte-enriched cultures: NPCs are differentiated in planar culture in astrocyte medium (ScienCell). Astrocytes are defined by GFAP and S100β (cultures contain 90% S100β+ and 82% GFAP+ by flow cytometry). Cortical neuron differentiation: For differentiation,65 NPCs are dissociated by Accutase and plated onto poly-D lysine- and LMN-coated dishes. Cortical neuronal differentiation medium consists of 1:1 DMEM/F12: Neurobasal, 1% N2 supplement, 1% B27 supplement, 1% L-glutamine 2 mM, 0.5 mg/ml bovine albumin fraction V, 55 μM β-mercaptoethanol and 8.6 mM glucose, with feeding 3-4 times per week. Neurons are assessed at 6 weeks in culture. Pericytes: Primary human brain pericytes are purchased directly from ScienCell and propagated in ScienCell Pericyte Medium.

Tumor cell acquisition and processing. We will investigate infiltrating gliomas with a focus on GBMs. Biospecimens will be obtained in cooperation the Ohio State University Comprehensive Cancer Center (OSUCCC) Tissue Procurement Core (TPC) (IRB protocol 2019C0196, Biorepository of Patient-Derived Tumor Models) and a new IRB protocol in preparation by Drs. Skardal and Elder. Dr. Elder will serve as facilitator between the clinical and laboratory research environments. We will obtain at least 45 patient biopsies (~9/year). As a reference, over the past several years Dr. Skardal generated a pipeline for transferring biospecimens of various tumor types from the clinic to lab, resulting in over 30 glioma biospecimens being transferred into PTO-based studies in less than 1 year. Should the workflow require additional samples, we do not anticipate a problem increasing accrual based on the clinical load at OSUCCC. The Skardal lab has biobanked cell suspensions and organoids from these biospecimens that can be used. All specimens are coded and deidentified, placed in RPMI (Roswell Park Memorial Institute) 1640 media, and then transferred through the TPC to the Skardal research team. Biospecimens are minced, washed, and incubated with collagenase and hyaluronidase to digest the ECM, as described. Portions will be preserved for histology and RNA extraction. Subsequent cell suspensions undergo dead cell removal and filtration prior to use.

CVU-T biofabrication: The conventional use of lithographically-defined PDMS elastomer for microfluidic devices is tedious due to reliance on expensive transparency masks, photolithography, serial casting, and precise layer alignment. However, this is necessary only for extraordinary resolution. Our 3D cell cultures enable the use of somewhat larger-scale, but considerably simpler thin, patterned adhesive films and poly(methyl methacrylate) (PMMA) layers that can be aligned and layered by folding and stacking to form microfluidic structures. This removes the need for a cleanroom or precise alignment and can be achieved by a computer-controlled laser cutter. TEER sensing will be integrated to these devices as previously described for continuous quantification. Notably, we will be employing a recent improvement to the physiological relevance of the BBB by moving from a planar model to using a tubular platform within a pump-optional microfluidic device (FIG. 7A-B) fabricated by 3D digital light processing (DLP, Cellink LUMEN X)) biofabrication (FIG. 7C), evolved from our extensive bioprinting experience. DLP printing will create a 3D ECM structure in which neural cells will be encapsulated, after which ECs will be seeded within the lumen. To do this, the tubular lumen structure will be “pulled” out of the hydrogel precursor reservoir with cells embedded, after which ECs will be seeded in the device to line the lumen walls. The PTO is embedded in the 3D volume of the CVU-T by first placing the PTO on predetermined locations on the reservoir surface. Thus, as the 3D lumen structure is formed, the PTO becomes embedded as the structure is formed.

ECM optimization for BBB function and characterization: ECM modulation. Thiolated HA and gelatin (Advanced Biomatrix) are dissolved 1% w/v, while a polyethylene glycol diacrylate (PEGDA, 3.4 kDa) crosslinker is dissolved at 2% w/v, in DI water containing 0.1% w/v photo-initiator (2-Hydroxy-4′-(2-hydroxyethoxy)-2-methylpropiophenone). These are mixed in a 2:2:1 ratio by volume and supplemented with thiolated FN, LMN, or collagen III and IV (modified in house through maleimide-thiol chemistry) at an end concentration of 0.25 μg/ml each. Pericytes, astrocytes, and neurons are suspended in the hydrogel precursor in density of 2×10⁷ cells/ml each and are crosslinked by UV light (1 s with intensity of 1 W/cm2 ). Modulation will primarily entail increases or decreases of the gelatin, FN, and LMN components by an order of magnitude, testing combinations of these 3 ECM components. Our aim is to maintain the HA structural component concentration to retain an elastic modulus in the range of brain tissue stiffness (400 to 2000 Pa). BBB integrity assays: Immunostaining with the following biomarkers will qualitatively assess the CVU construct: CD31, ZO-1, N-cadherin, GFAP, MAP2, GLUT1, beta-III-tubulin, and S100b. Macro-confocal microscopy (Leica TCI LSI) will be employed to provide visualization in 3D of interactions. Electrodes will be fabricated to fit CVU devices as in previous studies. Measurements are documented from readings using an Epithelial Volt/Ohm TEER Meter (WPIINC). Upon fabrication of the BBB, resistance will be low, increasing until reaching a plateau of EC confluency. Cell viability will be verified by LIVE/DEAD staining (Invitrogen). 70 kDa or 3 kDa FITC dextran in cell culture media will be added into the systemic device circulation. BBB transport will be determined by measuring fluorescence intensity of media aliquots in the brain fluid circuits. 3 kDa FITC dextran should pass through easily; 70 kDa should not. Only after increasing BBB permeability with histamine should the 70 kDa FITC dextran pass. Active transport will be assessed by studying activity of two efflux transport proteins commonly found in the BBB, p-glycoprotein (PgP) and multidrug resistant protein (MRP). Rhomadime 123 and H2DCFDA are fluorescent substrates of PgP and MRP respectively, so activity and directionality of the proteins can be easily quantified as previously described.

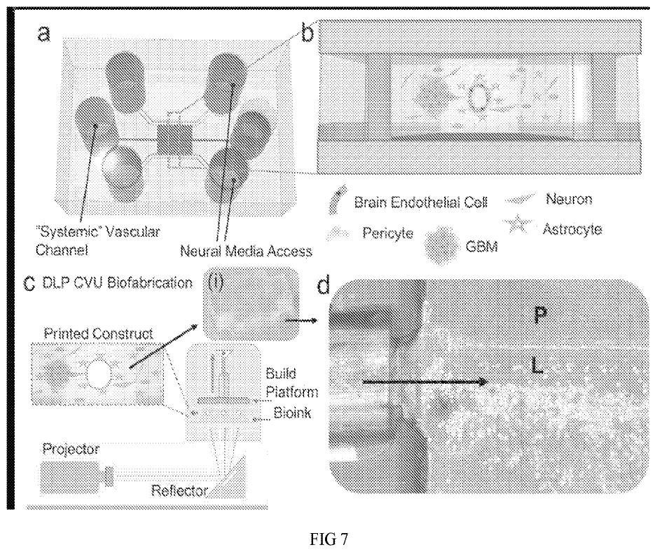

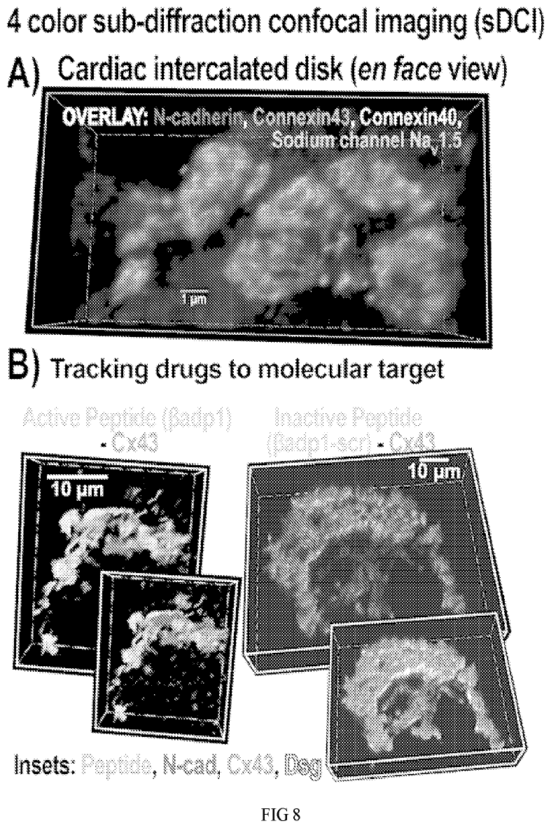

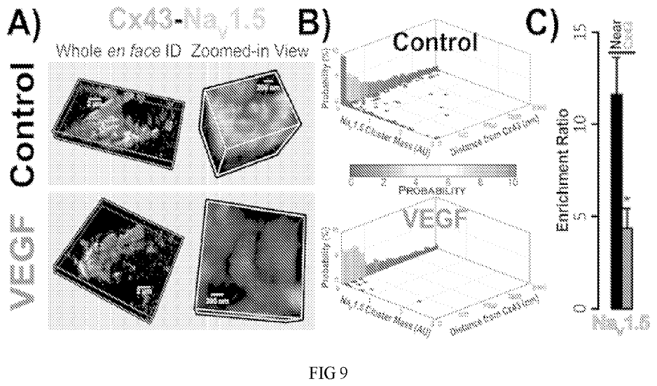

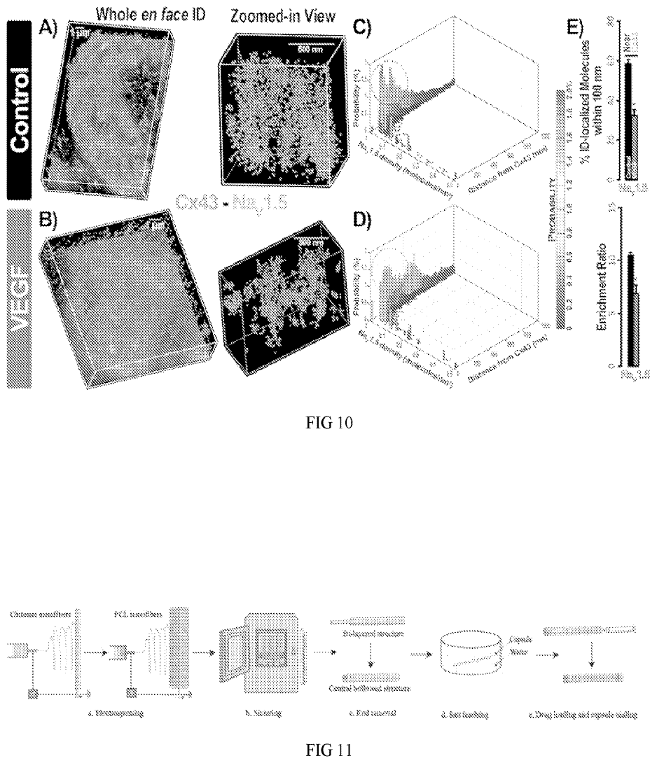

Quantitative assessment of CVU-T cellular and molecular structure: Spatial organization of cells within CVU-Ts will be assessed in 3D using sub-diffraction confocal microscopy (sDCI; FIG. 8-9), enabling simultaneous visualization of 4 spectral channels at 130 nm resolution. Resulting images analyzed using two approaches developed in the Veeraraghavan lab; Object-based Segmentation in 3D (OBS 3D) and Morphological Object Localization (MOL) will quantify spatial organization of different cell types relative to each other as cumulative distribution functions of inter-cell type distances, enabling rich, quantitative assessment conducive to robust hypothesis testing (2-sample Kolmogorov-Smirnov tests to compare whole distributions, Wilcoxon's test to compare central tendencies). Additionally, these approaches reveal spatial distribution of key proteins relative to cell-type markers and cellular landmarks (nuclei, cell periphery) within CVU-Ts. Proteins assessed will include Cx43, its scaffolding protein ZO-1 and mechanical junction proteins (N-cadherin, desmoglein 2, integrin β1). Furthermore, the spatial distribution of proteins identified at the cell surface and cell-cell contacts will be assessed at higher resolution with TSTED (fluorescence lifetime imaging-couplined STimulated Emission Depletion microscopy; simultaneous visualization of up to 2 spectral channels at <40 nm resolution with additional confocal channels; FIG. 9). MOL analysis of sDCI and τSTED images quantify distribution of key cell types and proteins within CVU-Ts. Finally, the nanoscale distribution of therapeutic target Cx43 will be assessed relative to spatial landmark proteins (mechanical junction proteins identified at cell-cell contacts, particularly in GSCs) using STochastic Optical Reconstruction (STORM) microscopy (simultaneous localization of up to 2 protein species at 20 nm resolution). STORM (FIG. 10) will provide orthogonal validation of τSTED results plus additional information on molecular density. STORM single molecule localization data will be processed using machine learning-based cluster analysis [STORM-RLA] to quantitative 3D map of therapeutic targets within CVU-Ts, which are utilized in additional examples to assess treatment efficacy.

### Example 5 In Vitro Tuning of Microcapsule-Based CT11 Release Kinetics and TMZ Dosing in 3D Patient-Derived GBM Tumor Organoids of Individual and Mixed GBM Subpopulations

In vitro tuning of microcapsule-based release of αCT11 for sustained long-term release (>6 months) and tumor killing efficacy when delivered with TMZ through microcapsule design and testing in culture with GBM PTOs (no CVU-T).

Microcapsule fabrication: Microcapsules are fabricated using a modified version of our published protocol, as shown in FIG. 11. Briefly, the inner chitosan fibrous layer is prepared by electrospinning 5% w/v chitosan dissolved in trifluoroacetic acid: dichloromethane (TFA: DCM) onto a 1 mm diameter stainless steel rotary collection rod. A nanoporous layer of PCL is then deposited onto the chitosan layer by electrospinning PCL with salts. Briefly, a combination of PCL and HEPES sodium salts are dissolved in hexafluoroisopropanol (HFP). Following electrospinning, the bilayered cylinders will be sintered under vacuum at 100° C. 3 hours to melt the outer PCL layer and remove surface porosity. Sintered cylinders are then washed in saturated sodium bicarbonate solution and DI H₂O to remove the water soluble HEPES sodium salts, leaving nanopores in the sintered capsule.

To reduce porosity to sustain release of the lower MW αCT11 (1.6 kDa) approximately 6 months, four mass ratios of PCL: HEPES will be evaluated (95:5, 96:4, 97:3, 98:2) to assess the impact of salt concentration on porosity and release. Capsule inner and outer diameter will be measured using a digital micrometer, and will be verified by microscopy. SEM will be used to image the capsules and quantify average pore diameter, as previously described, quantifying >100 pores from at least 3 images using ImageJ.

αCT11 release kinetics studies: Capsules prepared as above are be loaded with FITC-labeled αCT11 dissolved in DMSO and PBS at a concentration of 2 mM. Briefly, after vacuum drying, capsules will be cut to 7 mm length and sealed on one end with a heat sealer (Doug Care Equipment, TTS-8C) as published. Dissolved therapeutic will be loaded into the capsule via 31G syringe needle. The other end will be sealed, resulting in a 5 mm capsule due to 1 mm loss at each end from sealing. The mass of each capsule will be measured prior to study initiation. In vitro release will be determined as published. Briefly, capsules will be incubated at 37° C. in low binding centrifuge tubes with 2.0 ml PBS. At 1 day, 3 days, 1 week, 2 weeks, 1 month, and monthly thereafter to at least 6 months, eluent will be collected and 2.0 ml fresh PBS will be added to maintain sink conditions. Release rate of FITC-labeled therapeutic will be evaluated using fluorescent plate reader at 525 nm (with 488 nm excitation). Therapeutic bioactivity will be assessed at each time point as described below in dosing studies. At the end of the study, capsules will be retrieved and then vacuum dried for characterization. SEM will be used to analyze capsule size, pore size, and defects, and ImageJ will be used to compare with initial capsule and pore sizes prior to incubation.

Generation of individual versus mixed population GBM organoids: While GBM subtypes have been classified, the delineations between subtypes have not translated in to changes in clinical practice. We have generated ex vivo cell populations corresponding with these subtypes for PTO biofabrication. We can isolate the GSC population, which is often resistant to TMZ—and other therapies—and drives tumor recurrence, and biofabricate GSC-biased PTOs. Following biospecimen processing, the cell suspension is sorted by antibody-functionalized magnetic beads (rather than flow cytometry to increase cell viability) according to surface markers in a particular order. Specifically, CD133 selects for GBM stem cells (GSCs), CD44 selects for the mesenchymal subtype, PDGFRA selects for the proneural subtype, EGFR (without PDGFRA) selects for the classical subtype, and remaining cells representing what the field previously regarded as the neural subtype. Tumor cells are fluorescently labeled prior to use. PTOs are created using the same ECM hydrogel system as described. PTOs are each 10 μl volumes of 2×10⁷ cells/ml hydrogel precursor solutions deposited into 48-well plates by a bioprinter (Cellink Bio X) for dosing studies to accommodate mirocapsules.

BBB-free TMZ-αCT11 dosing studies. In our preliminary studies, we have shown efficacy of TMZ-αCT11 combinatorial treatment in 3D GBM cell line organoids with some TMZ resistance. We will repeat this study and perform additional dosing studies with GBM PTOs (no CVU) comprised of individual subpopulations and the entire heterogeneity of the biospecimens. First, PTOs in 48-well plates will be treated with TMZ only, TMZ+αCT11, TMZ+αCT11-loaded microcapsule, or no treatment while varying TMZ concentrations and holding αCT11 constant. These will be assessed for viability at 1 day, 3 days, 1 week, 2 weeks, 1 month, and monthly thereafter. Second, PTOs will be treated in the same groups while holding TMZ constant and varying αCT11 concentrations. These studies will help optimize target half maximal effective concentration (EC50)84 values for each drug, which will further inform loading and release kinetic requirements. Assessment of viability and drug efficacy will be determined by quantification of ATP activity (CellTiter Glo 3D, Promega) and LIVE/DEAD staining (Thermo Fisher) with confocal imaging and unbiased quantification of viable versus dead cells. Additionally, TMZ+αCT11 promotes autophagy in GSCs via AKT/mTOR activation. Thus, we will assess autophagy (Cyto-ID assay; Enzo Life Sciences) and AKT/mTOR activation in order to verify treatment efficacy and mode of action.

BBB-free TMZ and microcapsule-based aCT11 dosing studies. Dosing studies will be performed once more, but as a validation of microcapsule-based sustained release of αCT11. αCT11-loaded and αCT11-free microcapsules will be encapsulated at the same time that cells are encapsulated within the ECM hydrogels to form PTOs, thus immobilizing the microcapsules. It is known that αCT11 is effective with one-time dosing at 100 μM but the therapeutic level in this context and with sustained release at lower levels has yet to be determined. To make these measurements, these PTOs will be treated with TMZ that will be replenished periodically during long-term treatment cycles (28 and 56 days). PTOs will be sacrificed every 7 days and evaluated for viability as described above to quantify efficacy and an EC50 equivalent of the combinatorial treatment using the fluorescence measurements and imaging described above.

### Example 6: αCT11-TMZ Combinatorial Treatment in 3D CVU-T Devices Containing Patient-Derived GBM Tumor Organoids

The CVU-T platform containing GBM PTOs is used to evaluate tumor cell killing efficacy of combinatorial microcapsule-based localized, sustained delivery of αCT11 with systemic TMZ delivery, using super-resolution microscopy to assess successful molecular targeting and efficacy of the drug treatment.

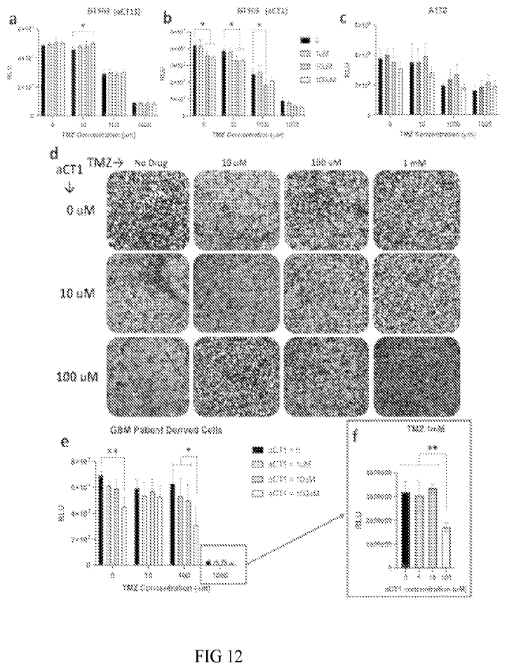

TMZ-αCT11 combinatorial treatment in GBM organoids: TMZ and αCT11 have been tested together against GBM cells in simple 2D cell cultures with a single GBM cell line, demonstrating improved TMZ efficacy. To build on these 2D studies we created GBM organoids from the GBM cell line A172, the GSC line BT169, and a heterogeneous patient-derived GBM cell population, and assessed whether or not αCT1 or αCT11 improved TMZ efficacy in 3D. FIG. 12 shows organoid responses via quantitative ATP activity following 8 days of treatment, and LIVE/DEAD fluorescent imaging data for the BT169 organoids. In GSC BT169 organoids, αCT11 did not enhance TMZ-based tumor cell killing, while αCT1 did (FIG. 12A-12B). A172 organoids showed a dose dependent response to TMZ, with little enhancement with αCT1 (FIG. 12C; αCT11 not shown, but similar result). The response of BT169 organoids was also visualized through LIVE/DEAD staining (FIG. 12D). Next, we utilized GBM PTOs as a test of heterogeneous tumor cell populations with this therapeutic approach. αCT1 did significantly increase TMZ efficacy (FIG. 12E-12F). These data suggest that the αCT1 peptide, which has a cell penetration amino acid sequence, may be necessary for entry of the peptide into the cell.

Combinatorial TMZ and αCT11 treatment in CVU-Ts: Combinatorial TMZ-αCT11 treatment in studies in CVU-Ts are initiated by incorporating the αCT11 microcapsule added in the parenchymal 3D CVU volume, embedded within the hydrogel upon DLP biofabrication. This is performed by placing microcapsules at predetermined locations on the DLP bioprinter build surface corresponding with the 3D digital CVU-T architecture. During bioprinting, the microcapsules become crosslinked into the parenchymal volume of the printed structures, as described for PTOs. Two top performing microcapsule designs will be prepared and loaded with αCT11 as described above (n=5). Blank microcapsules and therapeutic without encapsulation will be used as controls. Studies will use published techniques. At the termination of the studies, microcapsules will be retrieved from the CVU-Ts, and remaining therapeutic will be quantified by fluorescence after breaking capsules and washing with known volumes of PBS. This will enable calculation of therapeutic loading efficiency and percent released at each time point. Further, microcapsules will be imaged using SEM to evaluate changes in porosity and wall thickness to assess in vitro biodegradation and to check for any defects in the device. For drug treatment durations, we expect based on preliminary αCT11 release kinetics (FIG. 6) that 6 months of sustained αCT11 release is a realistic goal. Rather, our initial goal to perform 2, 4, 6, and 8 week TMZ-αCT11 treatment regimens with corresponding analyses, potentially tuning TMZ administrations with αCT11 release profile data. We have previously maintained both individual organoid and organ-on-a-chip systems, as well as 3- and 6 -tissue organ-on-a-chip methods for at least 4 weeks with no major hurdles encountered. TMZ will be administered at 10 uM (expected; based on prelim data from FIG. 6 and previous PTO studies), delivered into the media reservoirs of the CVU-T devices, and be refreshed every 7 days.

Evaluation of therapeutic efficacy by traditional and super-resolution imaging assays Therapeutic bioactivity will be measured as described, but adapted to the CVU-T. LIVE/DEAD stains will be imaged on chip by confocal microscopy and cross-referencing the labeled PTO cells with the LIVE/DEAD calcein AM and ethidium homodimer-1 dyes. Co-localization with the PTO cell label positively identifies a GBM tumor cells, while lack of the label identifies a non-tumor neural cell of the CVU-T. If such cells fluoresce red, this would indicate off target toxicity, an unwanted side effect. Super-resolution imaging approaches (sDCI, τSTED, STORM) will be used to acquire 3D Z-stacks of entire CVU-Ts. Fluorescently-labeled αCT11 distribution within CVU-Ts will be assessed in relation to its molecular target (Cx43) and other relevant proteins (N-cadherin, Desmoglein), cellular landmarks (cell periphery, nuclei), and cell type-specific markers. As described above, while imperfect, CD133, CD44, PDGFRA, and EGFR roughly correspond to the GSC, mesenchymal, proneural, and classic GBM molecular subtypes. While, new methods of subtyping are underway, because these more established subtypes do have specific genotypic and phenotypic differences, we assess how treatment impacts each subpopulation. OBS3D and MOI analysis of sDCI, τSTED images will enable the assessment of αCT11 peptide distribution throughout the volume of CVU-Ts in relation to cell-type markers and key cell-cell contact proteins. This will reveal how the therapy targets individual GBM subpopulations and validate peptide delivery to target-rich cell membrane/cell-cell contact niches within them. Additionally, STORM-RLA analysis of single molecule localization data enable robust quantitative assessment (normalized relative spatial density of molecules) of αCT11 peptide distribution relative to its target, Cx43.

Tumor organoid maintenance of genomic profile: RNAseq and hierarchical cluster analysis performed as described in FIG. 4 on tumor regions extracted from CVU-Ts in a verification step to determine the PTOs' and CVU-Ts' maintenance of the genomic profile of the originating tumor. Because the PTOs are labeled, CVU-Ts can be treated like tissue biospecimens and be dissociated w/collagenase/hyaluronidase and dispase as described. Labeled glioma cells will be isolated by FACS for RNAseq and compared with that of the originating biospecimen. We isolate RNA (Qiagen RNeasy) and RNAseq performed for the populations in question (OSUCCC Genomic Shared Resource), with dataset alignment performed at the Ohio Supercomputer Center, and analyzed with our bioinformatics core. In particular, we are interested relative expression changes of genes associated with the GSC, mesenchymal, proneural, and classic subpopulations (e.g., CD133, CD44, PDGFRA, EGFR). For more breadth, we perform hierarchical cluster analysis (R, R-Project) of the 1000 most variably expressed genes to verify continued maintenance of the original tumor genomic profiles in the CVU-Ts (i.e., FIG. 4).

While the invention has been described with respect to specific examples including presently preferred modes of carrying out the invention, those skilled in the art will appreciate that there are numerous variations and permutations of the above described systems and techniques that fall within the spirit and scope of the invention as set forth in the appended claims.

1. A biodegradable microcapsule device comprising
a luminal core compartment comprising a medication or adjuvant;
an inner layer of biodegradable polymers surrounding the core;
an outer layer surrounding and adhered to the inner layer;
nanopores throughout the microcapsule;
the microcapsule being sealed.

2. The microcapsule of claim 1, wherein the inner layer comprises chitosan nanofibers, chitosan, a polyethyleneimine, a protamine, a polypropylimine, a poly-L-lysine, a poly-L-arginine, a poly-D-lysine, a poly-D-arginine, a cellulose, a dextran, a poly(amidoamine), poly(2-(dimethylamino)ethyl methacrylate), derivatives thereof, or combinations thereof.

3. The microcapsule of claim 1, wherein the outer layer comprises PCL nanofibers, a poly(e-caprolactone) (PCL), a poly-lactic acid (PLA), a poly-glycolic acid (PGA), a poly-lactide-co-glycolide (PLGA), a polyester, a poly(ortho ester), a poly(phosphazine), a poly(phosphate ester), a gelatin, a collagen, a polyethylene glycol (PEG), derivatives thereof, or combinations thereof.

4. The microcapsule of claim 1, wherein the medication or adjuvant is a selective connexin hemichannel inhibitor.

5. The microcapsule of claim 4, wherein the medication or adjuvant is αCT11 or αCT1.

6. The microcapsule of claim 1, wherein the microcapsule comprises more than 100 nanopores, each nanopore between about 10 and about 1000 nm in diameter.

7. The microcapsule of claim 1, wherein the microcapsule is approximately 5 to 20 mm in length.

8. The microcapsule of claim 1, wherein the microcapsule is approximately 5 to 7 mm in length with a diameter of about 0.5 mm to about 2 mm.

9. The microcapsule of claim 1, wherein the microcapsule is in the shape of a wafer, a tube, or a cylinder.

10. The microcapsule of claim 1, wherein the total amount of medication or adjuvant contained in the capsule and the rate at which the medication or adjuvant is released from the capsule and the size of the microcapsule are tunable to a specific subject and condition for which the microcapsule is used.

11. The microcapsule of claim 1, wherein the medication or adjuvant comprises a combination of two or more medications or adjuvants.

12. A method of making a microcapsule comprising:
providing means to form a luminal core compartment;
making an inner layer by collecting chitosan nanofibers, chitosan, a polyethyleneimine, a protamine, a polypropylimine, a poly-L-lysine, a poly-L-arginine, a poly-D-lysine, a poly-D-arginine, a cellulose, a dextran, a poly(amidoamine), poly(2-(dimethylamino)ethyl methacrylate), derivatives thereof, or combinations thereof on the luminal core means by electrospinning;
making an outer layer by collected PCL nanofibers, a poly(e-caprolactone) (PCL), a poly-lactic acid (PLA), a poly-glycolic acid (PGA), a poly-lactide-co-glycolide (PLGA), a polyester, a poly(ortho ester), a poly(phosphazine), a poly(phosphate ester), a gelatin, a collagen, a polyethylene glycol (PEG), derivatives thereof, or combinations thereof on the inner layer by electrospinning;
sintering the electrospun materials to remove macroscale pores;
removing the luminal core means, creating a hollow luminal core compartment;
salt leaching the hollow microcapsule to create a nonporous structure;
sealing any open edges of the hollow microcapsule,
inserting a medication or adjuvant into the hollow microcapsule;
sealing any port opened by the insertion of the medication or adjuant, thereby enclosing the medication or adjuvant within the inner and outer layers and forming a microcapsule.

13. The method of claim 12, wherein the means to form a luminal core compartment is a rod and the microcapsule formed on the rod is in the shape of a cylinder.

14. The method of claim 12 wherein the medication or adjuvant is a connexin hemichannel inhibitor.

15. A method of administering a medication or adjuvant to a subject in need of treatment, the method comprising:
implanting in or proximal to an area in need of treatment one or more biodegradable microcapsule device loaded with a medication or adjuvant, the microcapsule providing sustained release of the medication or adjuvant proximal to an area in need of treatment for a period of time

16. The method of claim 15, wherein the area in need of treatment is a cardiac tissue, muscle tissue, nervous tissue, connective tissue, epithelial tissue, a cartilage, a bone tissue, an adipose tissue.

17. The method of claim 15, wherein the medication or adjuvant is a selective connexin hemichannel inhibitor.

18. A method of administering to a subject in need to treatment for a cancer a therapeutic, the method comprising:
coincident with surgical cancer removal from the subject, implanting in a tumoral area at least one biodegradable microcapsule device loaded with a medication or adjuvant, the microcapsule providing sustained release of the medication or adjuvant to the tumoral area for a period of time; and
treating the subject post-surgically with a therapeutic that crosses the blood brain barrier.

19. The method of claim 18 wherein two or more biodegradable microcapsule devices loaded with a medication or adjuvant are implanted coincident with surgical cancer removal.

20. The method of claim 18, wherein the cancer is a glioblastoma.

21. The method of claim 18, wherein the therapeutic is TMZ.

22. The method of claim 18, wherein the microcapsule releases a connexin hemichannel inhibitor continuously for two, four or six months.

23. The method of claim 18, wherein the medication or adjuvant is a selective connexin hemichannel inhibitor.

24. The method of claim 23, wherein the connexin hemichannel inhibitor is a peptide mimetic.

25. The method of claim 23, wherein the connexin hemichannel inhibitor is αCT11 or αCT1.

26. The method of claim 18, wherein rate of release of the medication or adjuvant from the microcapsule is adjustable to be specific to the subject or the cancer that is being treated.

27. The method of claim 18, wherein the amount of connexin hemichannel inhibitor loaded into the microcapsule and the rate at which the connexin hemichannel inhibitor is released from the microcapsule is specific for the subject or a characteristic of the cancer to be treated.
